(19) United States
(12) Patent Application Publication
(10) Pub. No.: US 2026/0273740 A1
(43) Pub. Date: Sep. 17, 2026

(54) SYSTEMS AND METHODS FOR DETERMINING INSTRUMENT POSITION IN THE PRESENCE OF BACKLASH

(71) Applicant: INTUITIVE SURGICAL OPERATIONS, INC., Sunnyvale, CA (US)

(72) Inventor: Gabriel F. Brisson, Livermore, CA (US)

(21) Appl. No.: 19/565,214

(22) Filed: Mar. 12, 2026

Related U.S. Application Data
(60) Provisional application No. 63/771,456, filed on Mar. 13, 2025.

(51) Int. Cl. B25J 9/16; A61B 1/00; A61B 1/05; A61B 34/00

(52) U.S. Cl. CPC B25J 9/1641; A61B 1/00006; A61B 1/0005; A61B 1/00149; A61B 1/05; A61B 34/70; B25J 9/1633

(57) ABSTRACT

A robotically-assisted manipulator system includes an instrument, an instrument manipulator, and a controller. The instrument includes first and second instrument drivers coupled such that movement of the first instrument driver causes movement of the second instrument driver. The instrument manipulator includes first and second carriage drivers and a sensor. The first and second carriage drivers couple to the first and second instrument drivers. The controller is configured to: apply a first torque to the first carriage driver configured to impart motion to the first instrument driver to move the instrument in a degree-of-freedom; apply a second torque to the second carriage driver; adjust a magnitude of the first torque to cause a desired degree of movement of the instrument; receive a sensor signal indicating a position of the second carriage driver; and determine, based on the position of the second carriage driver, a current instrument position.

### CROSS-REFERENCED APPLICATIONS

This application claims priority to and benefit of U.S. Provisional Application No. 63/771,456 filed Mar. 13, 2025 and entitled “Systems and Methods for Determining Instrument Position in the Presence of Backlash,” which is incorporated by reference herein in its entirety.

### FIELD

The present disclosure is directed to systems and methods for determining a position of a portion of an instrument in the presence of backlash in a drive system controlling movement of the instrument. More specifically, backlash in various components of an instrument drive system tends to introduce error into position calculation and the systems and methods disclosed herein reduce the effects of backlash and improve accuracy in determining instrument position.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide the clinician with an image of a field of view within the patient anatomy. Some minimally invasive medical tools may be robot-assisted including teleoperated, remotely operated, or otherwise computer-assisted.

An instrument drive system configured to manipulate an instrument may experience backlash. For example, tolerances between teeth in a geared interface may introduce backlash into the instrument drive system which, in turn, may introduce error into tracking of a configuration of the instrument. This error can present challenges not only in tracking the absolute configuration of the instrument but also relative positioning and orientation between the instrument and other instruments being utilized, the patient, a pre-operative anatomical model, etc.

Accordingly, it is desirable to reduce the effects of backlash on instrument tracking in order to improve the accuracy of medical procedures.

### SUMMARY

The embodiments of the invention are best summarized by the claims that follow the description.

In an aspect of the present disclosure, a robotically-assisted manipulator system may include an instrument, an instrument manipulator, and a controller. The instrument may include a first instrument driver and a second instrument driver. The first and second instrument drivers may be coupled such that movement of the first instrument driver causes movement of the second instrument driver. The instrument manipulator may include a first carriage driver, a second carriage driver, and a sensor configured to monitor a position of the second carriage driver. The first carriage driver may be configured to couple to the first instrument driver and the second carriage driver may be configured to couple to the second instrument driver. The controller may be in operative communication with the instrument manipulator. The controller may be configured to: apply a first torque to the first carriage driver; apply a second torque to the second carriage driver; adjust a magnitude of the first torque to cause a desired degree of movement of the instrument in a degree-of-freedom; receive a sensor signal from the sensor indicating a position of the second carriage driver; and determine, based on the position of the second carriage driver, a current instrument position in the degree-of-freedom. The first carriage driver may be configured to impart motion to the first instrument driver to cause movement of the instrument in the degree-of-freedom. The magnitude of the first torque may compensate for at least one of an external force on the instrument or the second torque.

In some examples, the controller may be configured to apply the second torque to the second carriage driver when the desired degree of movement of the instrument in the degree-of-freedom is no movement. The controller may be configured to simultaneously apply the first torque to the first carriage driver and the second torque to the second carriage driver. When the desired degree of movement of the instrument in the degree-of-freedom is no movement, the magnitude of the first torque may be equal to a magnitude of the second torque.

In some examples, the first and second carriage drivers may each include a first engagement feature and the first and second instrument drivers may each include a second engagement feature configured to mate with the first engagement feature of a corresponding one of the first carriage driver or the second carriage driver. Application of the second torque to the second carriage driver may retain the first engagement feature of the second carriage driver in contact with the second engagement feature of the second instrument driver.

In some examples, the instrument may include a sterile adapter that includes the first and second instrument drivers. In some examples, a sterile adapter may be provided which includes first and second adapter drivers configured to be positioned between respective pairs of the first and second carriage drivers and the first and second instrument drivers.

In some examples, the controller may be configured to continuously apply the second torque to the second carriage driver at a constant magnitude and to vary at least one of the magnitude or a direction of the first torque to impart the desired degree of movement of the instrument in the degree-of-freedom. When the first torque and the second torque are applied in opposite directions, the magnitude of the first torque may be greater than a magnitude of the second torque to impart the motion to the first instrument driver. The controller may be configured to reduce the magnitude of the first torque to equal a magnitude of the second torque to retain the instrument stationary in the degree-of-freedom.

In some examples, the first instrument driver and the second instrument driver may be coupled to each other by an output shaft of the instrument. Each of the first and second instrument drivers may be independently coupled to the output shaft. The first instrument driver and the second instrument driver may be rotationally coupled by the output shaft. The first instrument driver, the second instrument driver, and the output shaft may each comprise a gear. Alternatively, the first instrument driver and the second instrument driver may be translationally coupled by the output shaft.

In some examples, the instrument may be a medical instrument that includes a shaft having a distal end configured to be inserted into a patient anatomy. An image capturing device may be disposed at the distal end of the shaft. The degree-of-freedom of the instrument may be roll about a longitudinal axis of the shaft. The controller may be further configured to register a pose of the medical instrument in an instrument reference frame to a surgical reference frame. The controller may be further configured to calculate a pose of one or more second instruments of the manipulator system relative to the medical instrument. The controller may be further configured to register an image generated by the medical instrument to a model reference frame. The controller may be further configured to overlay model information on the image generated by the medical instrument.

In some examples, the sensor may include an encoder configured to monitor a position of the second carriage driver.

In an aspect of the present disclosure, a robotically-assisted manipulator system may include an instrument, an instrument manipulator, and a controller. The instrument may include a first instrument driver and a second instrument driver. The first and second instrument drivers may be coupled such that movement of the first instrument driver causes movement of the second instrument driver. The instrument manipulator may include a first carriage driver, a second carriage driver, and a sensor configured to monitor a position of the second carriage driver. The first carriage driver may be configured to couple to the first instrument driver and the second carriage driver may be configured to couple to the second instrument driver. The controller may be in operative communication with the instrument manipulator. The controller may be configured to: apply a first torque to the first carriage driver, the first carriage driver configured to impart motion to the first instrument driver to cause movement of the instrument in a degree-of-freedom; receive a sensor signal from the sensor indicating a position of the second carriage driver; and determine, based on the position of the second carriage driver, a current instrument position in the degree-of-freedom.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates a schematic view of an example of a medical system in accordance with the present disclosure.

FIG. 2 illustrates a perspective view of an example of a manipulator assembly in accordance with the present disclosure.

FIG. 3 illustrates an example of an instrument in accordance with the present disclosure.

FIGS. 4-8 illustrate an example of a backend assembly at which a manipulator assembly engages an instrument in accordance with the present disclosure.

FIG. 9A illustrates an example of instrument drivers of a proximal control mechanism of an instrument in accordance with the present disclosure.

FIG. 9B illustrates a schematic view of interaction between instrument drivers and an output shaft in accordance with the present disclosure.

FIG. 9C illustrates an enlarged view of a portion of FIG. 9B.

FIG. 10 illustrates an example of a proximal control mechanism and sterile adapter in accordance with the present disclosure.

FIG. 11 illustrates a cross-sectional view of a backend assembly in accordance with the present disclosure.

FIGS. 12A-12B illustrate examples of methods for determining an instrument position in accordance with the present disclosure.

FIGS. 13A-13B illustrate an example of overlaying information on real-time image in accordance with the present disclosure.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

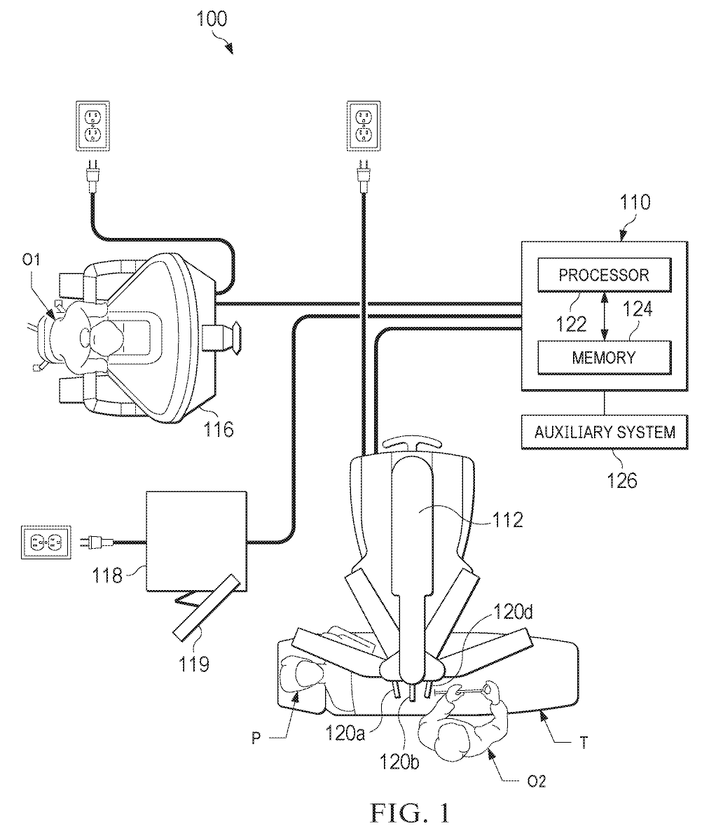

FIG. 1 provides an overview of a medical system 100 that may be used in, for example, medical procedures including diagnostic, therapeutic, or surgical procedures. In some examples, the medical system 100 may be a robot-assisted medical system that is under the teleoperational control of an operator O1 (e.g., a surgeon, a clinician, a physician, etc.). In alternative embodiments, the medical system 100 may be under the partial control of a computer programmed to perform the medical procedure or sub-procedure. In still other alternative embodiments, the medical system 100 may be a fully automated medical system that is under the full control of a computer programmed to perform the medical procedure or sub-procedure with the medical system 100. One example of the medical system 100 that may be used to implement the systems and techniques described in this disclosure is the da Vinci® Surgical System manufactured by Intuitive Surgical, Inc. of Sunnyvale, California.

As shown in FIG. 1, the medical system 100 may generally include a control system (or “controller”) 110, a manipulator assembly 112, an operator input system 116, and a component cart 118. In some examples, components of the control system 110 may be located in the manipulator assembly 112, the operator input system 116, and/or the component cart 118. The control system 110, manipulator assembly 112, operator input system 116, and component cart 118 may be coupled (e.g., via wired or wireless connections) so that the components together act as a single teleoperated minimally invasive medical system that provides an intuitive telepresence for the surgeon.

In one or more examples, the manipulator assembly 112 positioned near an operating table T on which a patient P is positioned may be a teleoperational assembly. The teleoperational assembly may be referred to as, for example, a teleoperational arm cart. One or more instruments 120 may be operably coupled to the manipulator assembly 112. In examples including a plurality of instruments 120, the plurality of medical instruments may include multiple of the same medical instrument and/or multiple different medical instruments. One or more of the medical instruments may comprise an endoscope.

The control system 110 may include at least one memory 124 and a processing unit with at least one processor 122 for effecting control between the manipulator assembly 112, the instruments 120, the operator input system 116, the component cart 118, and other auxiliary systems 126 which may include, for example, imaging systems, audio systems, fluid delivery systems, display systems, illumination systems, steering control systems, irrigation systems, and/or suction systems. The control system 110 also includes programmed instructions (e.g., stored on a non-transitory, computer-readable medium) to implement some or all of the methods described in accordance with aspects disclosed herein. While the control system 110 is shown as a single block in the simplified schematic of FIG. 1, the control system 110 may include multiple processors, memory devices, and/or circuits with a portion of the processing optionally being performed on or adjacent the manipulator assembly 112, another portion of the processing being performed at the operator input system 116, and/or another portion of the processing being performed at the component cart 118, and the like. Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one example, the control system 110 supports wireless communication protocols such as Bluetooth, IrDA, HomeRF, IEEE 802.11, DECT, and Wireless Telemetry.

In some examples, control system 110 may include one or more servo controllers that receive force and/or torque feedback from the instruments 120. Responsive to the feedback, the servo controllers transmit signals to the operator input system 116. The servo controller(s) may also transmit signals instructing manipulator assembly 112 to move the instruments 120 which extend into an internal surgical site within the patient body via openings in the body. Any suitable conventional or specialized servo controller may be used. A servo controller may be separate from, or integrated with, manipulator assembly 112.

The control system 110 may receive image data from an instrument 120 comprising an endoscopic imaging system and may process captured images (including still or video images) for subsequent display, such as to the operator O1 at the operator input system 116, the operator O2 viewing the display system 119, or on another suitable display located locally and/or remotely. For example, where a stereoscopic endoscope is used, the control system 110 can process the captured images to present the operator O1 with coordinated stereo images of the surgical site.

The operator input system 116 allows the operator O1 (e.g., a surgeon, a clinician, a physician, etc.) to view images of or representing the surgical site and to control the operation of the instruments 120. The operator input system 116 may be referred to as a control console. In some examples, the operator input system 116 may be located in a control environment which may be a non-sterile environment. In some examples, the control environment may be located in the same room as operating table T and, in some examples, the operator O1 and the operator input system 116 may be located in a different room or a completely different building from the patient P. The operator input system 116 generally includes one or more control device(s) for controlling the instruments 120. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same degrees of freedom as the instrument(s) 120 to provide the operator with telepresence, which is the perception that the control device(s) are integral with the instruments so that the operator has a strong sense of directly controlling instruments as if present at the surgical site. In other embodiments, the control device(s) may have more or fewer degrees of freedom than the associated instrument(s) and still provide the operator with telepresence. In some embodiments, the control devices are manual input devices that are movable with six degrees of freedom, and which may also include an actuatable handle for actuating instruments (for example, for closing grasping jaw end effectors, applying an electrical potential to an electrode, delivering a medicinal treatment, and actuating other types of instruments).

The operator input system 116 includes a display system which may include a stereoscopic display for presenting the operator O1 with a coordinated stereo view of the surgical environment that enables depth perception. In other embodiments, the display system of the operator input system 116 may include one or more other types of displays. The operator input system 116 may present images captured, for example, by an instrument 120 comprising an endoscope to display an endoscopic image of anatomic field of view in the patient P to the operator O1. The endoscopic image of the anatomic field of view may be augmented by graphical user interface elements such as virtual or synthetic menus, indicators, and/or other graphical or textual information to provide additional information to the viewer. The display system of the operator input system 116 may be a head-in display system in which the head of the operator O1 engages the operator input system 116 while viewing the displayed content.

The operator input system 116 further includes one or more input control devices which cause the manipulator assembly 112 to manipulate one or more instruments 120. The input control devices can provide the same degrees of freedom as their associated instruments to provide the operator O1 with telepresence, or the perception that the input control devices are integral with said instruments so that the operator has a strong sense of directly controlling the instruments. To this end, position, force, and tactile feedback sensors (not shown) may be employed to transmit position, force, and tactile sensations from the instruments 120 (e.g., surgical tools or endoscope), back to the operator's hands through the input control devices. Input control devices may include foot pedals that receive input from a user’s foot.

Component cart 118, which may also be referred to as a vision cart or tower, may include a display system 119 and may be located near or in the vicinity of the patient P, one or more clinical staff operator O2, and/or the manipulator assembly 112 in a patient environment. In some examples, some or all of the components in the patient environment may be sterilized or covered with sterilized drape. The clinical staff operator O2 may circulate within the environment and may access, for example, the manipulator assembly 112 during a local procedure and/or view the display system 119 from the patient bedside. The component cart 118 may house components of the control system 110 including, for example, a central electronic data processing unit. In some examples, the component cart 118 may include camera control units for the left and right image capture functions of an endoscope. The component cart 118 may further include optional auxiliary surgical equipment, such as electrosurgical units and insufflators. The display system 119 may display, for example, endoscopic images of the anatomic field of view, support content for reference by the operator O2, instructions for the operator O2, or other graphical user interface content that may be relevant to a medical procedure. In an alternative example, the display system 119 may be attached to the manipulator assembly 112 or may be a separate component in the patient environment disconnected from the component cart 118. In various examples, the display system 119 may include a monitor that may receive video input from the control system 110 or another image source (e.g., via HDMI connection).

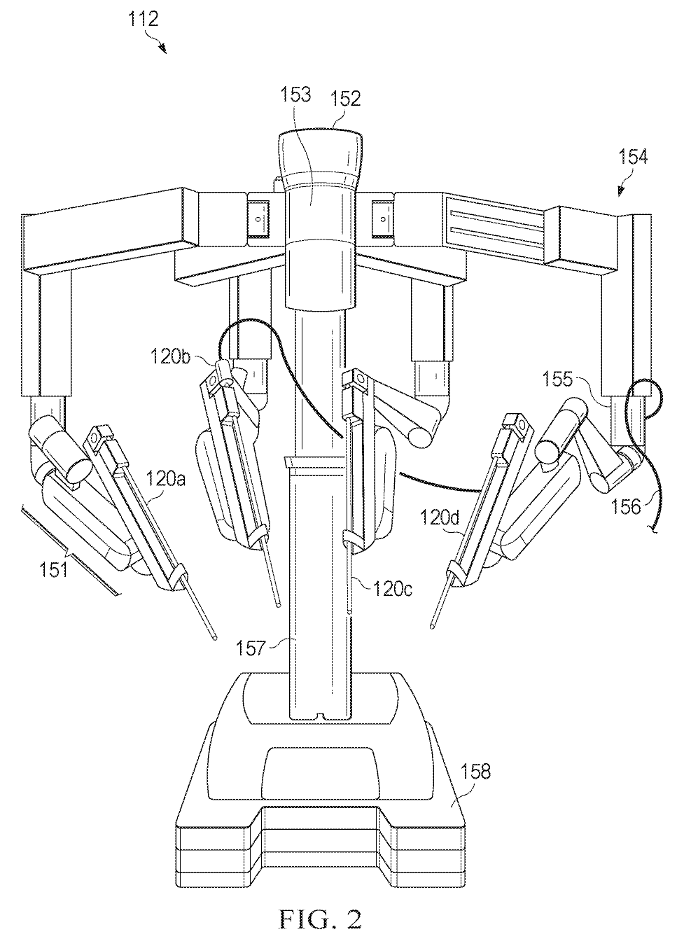

FIG. 2 is a perspective view of one embodiment of the manipulator assembly 112 which may be referred to as a patient side cart, surgical cart, teleoperational arm cart, manipulator assembly or surgical robot. The manipulator assembly 112 provides for the manipulation of one or more instruments 120 which, in the illustrated example, includes surgical tools 120a, 120c, and 120d and an endoscope 120b (“endoscopic imaging system”), such as a stereoscopic endoscope used for the capture of images of the site of the procedure. The endoscope 120b may transmit signals over a cable to the control system 110. Manipulation is provided by teleoperative mechanisms having a number of joints. The instruments 120a-120d may be positioned and manipulated through incisions in the patient so that a kinematic remote center is maintained at the incision to minimize the size of the incision. Images of the surgical site can include images of the distal ends of the surgical tools 120a, 120c, and 120d when they are positioned within the field of view of the endoscope 120b.

The manipulator assembly 112 includes a drivable base 158. The drivable base 158 is connected to a telescoping column 157, which allows for adjustment of the height of arms 154. The arms 154 may include a rotating joint 155 that both rotates and moves up and down. Each of the arms 154 may be connected to a rotatable orienting platform 153. The manipulator assembly 112 may also include a telescoping horizontal cantilever 152 for moving the orienting platform 153 in a horizontal direction. In this example, each of the arms 154 connects to a manipulator arm 151. The manipulator arms 151 may connect to an instrument 120. The manipulator arms 151 may be teleoperable. In some examples, the arms 154 connecting to the orienting platform 153 may not be teleoperable. Rather, such arms 154 may be positioned as desired before the operator O1 begins operation with the teleoperative components. Throughout a surgical procedure, instruments 120 may be removed and replaced with other instruments such that instrument to arm associations may change during the procedure.

An endoscopic imaging system (e.g., endoscope 120b) may be provided in a variety of configurations including a rigid or flexible endoscope. Rigid endoscopes include a rigid tube housing a relay lens system for transmitting an image from a distal end to a proximal end of the endoscope. Flexible endoscopes transmit images using one or more flexible optical fibers. Digital image-based endoscopes have a “chip on the tip” design in which a distal digital sensor such as a one or more charge-coupled device (CCD) or a complementary metal oxide semiconductor (CMOS) device store image data. Endoscopic imaging systems may provide two- or three- dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy.

The manipulator assembly 112 may support and manipulate the instruments while the operator O1 views the surgical site through the operator input system 116. An image of the surgical site may be obtained by the endoscope 120b, which may be manipulated by the manipulator assembly 112. The manipulator assembly 112 may include a kinematic structure of one or more non-servo controlled links (e.g., one or more links that may be manually positioned and locked in place, generally referred to as a set-up structure) and a manipulator. When the manipulator takes the form of a teleoperational manipulator, the manipulator assembly 112 is a teleoperational assembly. The manipulator assembly 112 may include a plurality of motors that drive inputs on the instruments 120. In some examples, these motors move in response to commands from the control system 110. The motors include drive systems which when coupled to an instrument 120 may advance the instrument through a naturally or surgically created anatomical orifice. Other motorized drive systems may move the distal end of an instrument 120 in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of an instrument 120 for grasping tissue in the jaws of a biopsy device or the like. Instruments 120 may include end effectors having a single working member such as a scalpel, a blunt blade, an optical fiber, or an electrode. Other end effectors may include, for example, forceps, graspers, scissors, or clip appliers.

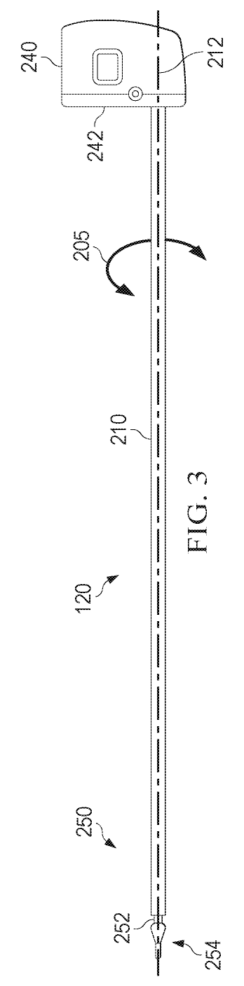

FIG. 3 illustrates an example of an instrument comprising a distal portion 250 and a proximal control mechanism 240 coupled by an elongate tube 210 (or “shaft”). The distal portion 250 of the instrument 120 includes an end effector 254. The end effector 254 may be any of a variety of medical tools, such as forceps, a needle driver, a cautery device, a cutting tool, an imaging device (e.g., an endoscope camera or ultrasound imaging device), or a combination that includes two or more tools. In the embodiment shown, the end effector 254 is coupled to the elongate tube 210 by a "wrist" 252 that allows the orientation of the end effector 254 to be manipulated with respect to the elongate tube 210. In some instruments, the wrist 252 may be omitted.

Some instruments 120 may include an end effector 254 that is controlled at least in part by a plurality of rods and/or flexible cables. Rods, which may be in the form of tubes, may be combined with cables to provide a "push/pull" control of the end effector 254 with cables providing flexible sections as required. A typical elongate tube 210 for an instrument 120 is small, perhaps five to eight millimeters in diameter. The cables must fit within the elongate tube 210 and be able to bend as they pass through the wrist 252.

Some instruments 120 may include an end effector 254 that is controlled by rotation of the shaft 210. That is, the shaft 210 may be rotatable in a degree-of-freedom 205 about a roll axis 212 with respect to the proximal control mechanism 240. For example, in an instrument 120 with an end effector 254 the comprises an endoscope, the shaft 210 may rotate about the roll axis 212 to rotate a view orientation.

The proximal control mechanism 240 of the instrument 120 includes an instrument interface 242. In use, the instrument 120 may be controlled by the manipulator assembly 112 through engagement of the instrument interface 242 with a corresponding carriage interface of a carriage of the respective manipulator arm 151.

In order to provide a sterile operation area, it is preferred that a barrier be placed between the non-sterile manipulator assembly 112 and the instrument(s) 120 in the sterile surgical field. Therefore, a sterile component, such as an instrument sterile adapter (ISA), may be placed between the instrument 120 and the manipulator arm 151. The placement of an instrument sterile adapter between the instrument 120 and the manipulator arm 151 ensures a sterile coupling point for the instrument 120. This permits removal and exchange of an instrument 120 from the manipulator arm 151 with another instrument 120 during the course of a procedure.

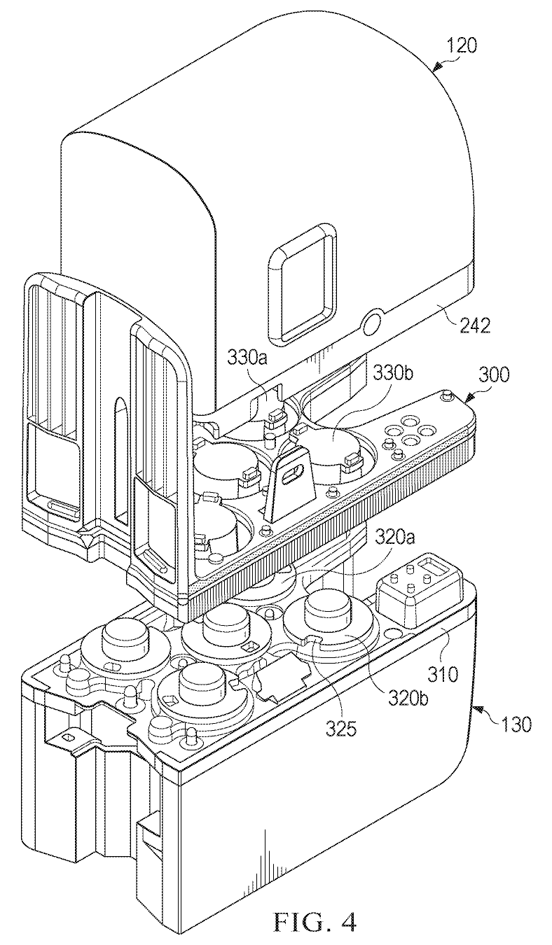

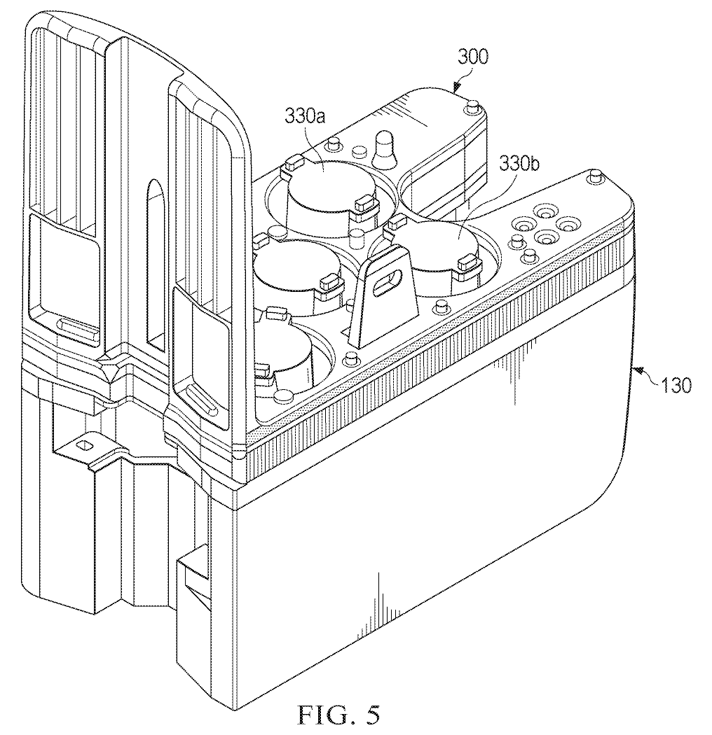

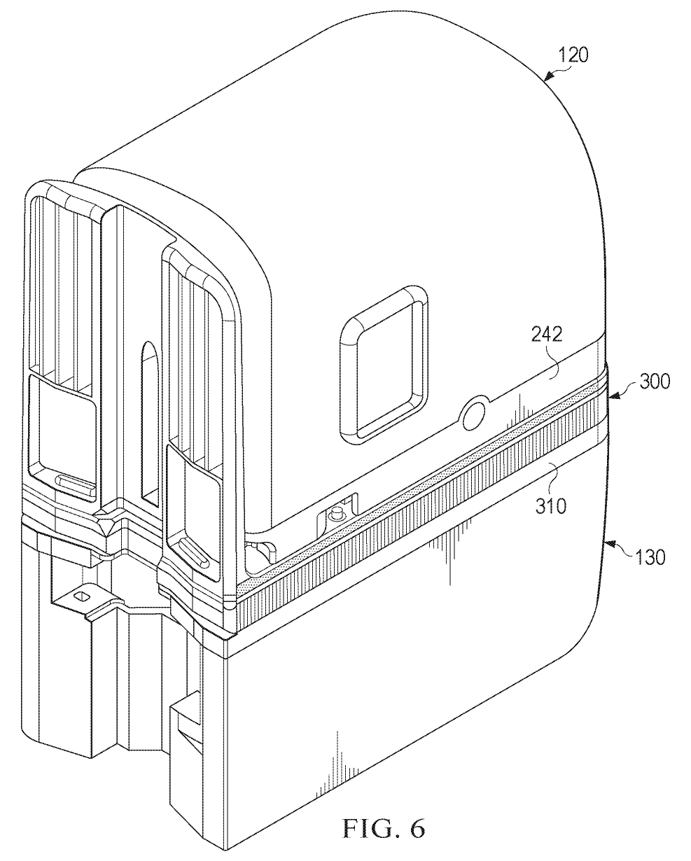

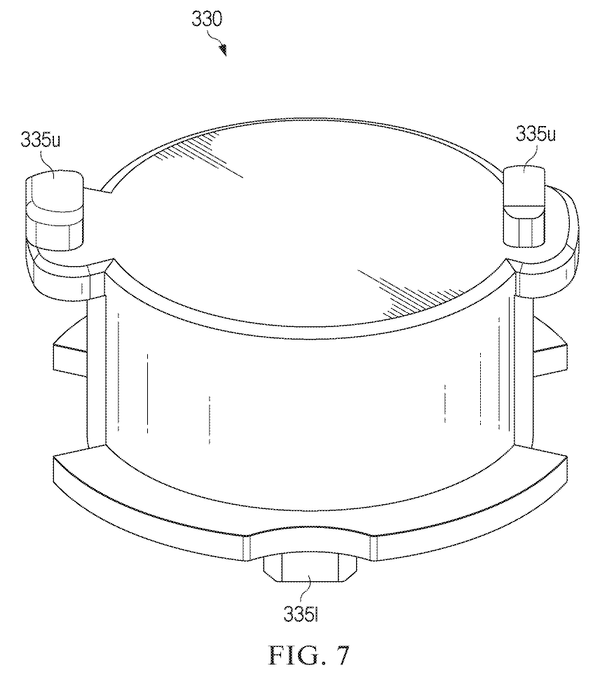

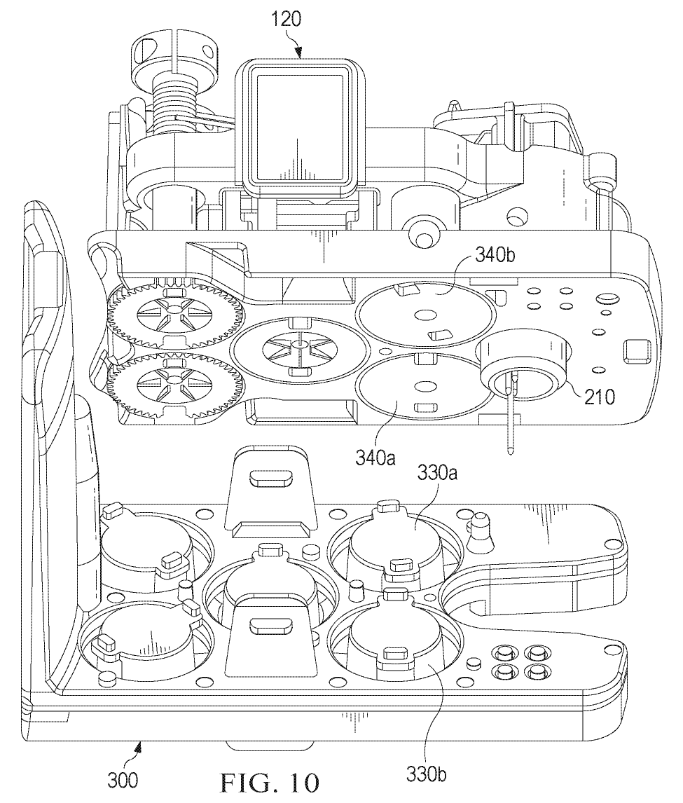

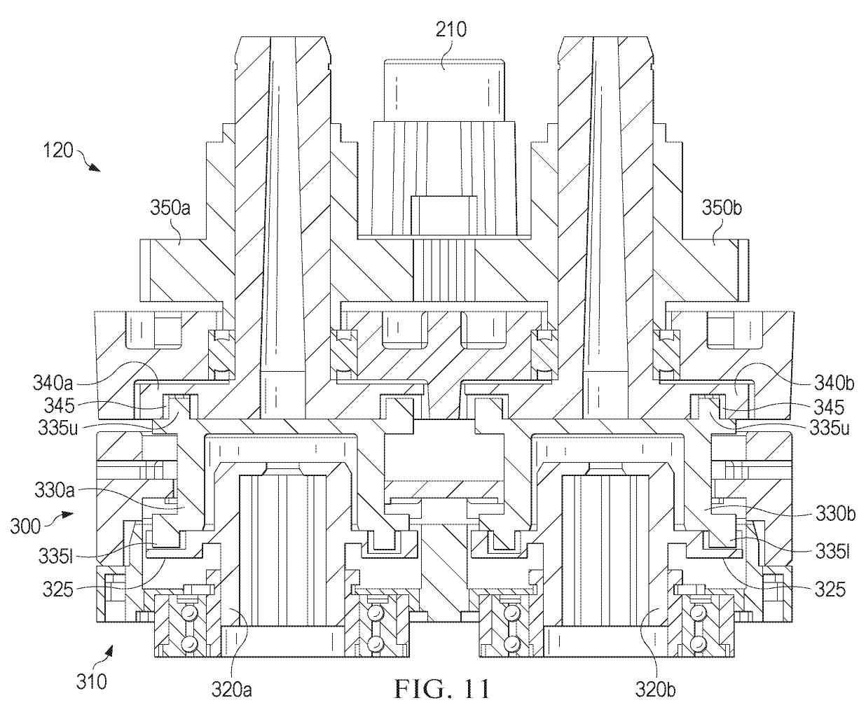

FIGS. 4-11 illustrate an example of a backend assembly at which the instrument interface 242 of the proximal control mechanism of an instrument 120 is engaged with the carriage interface 310 of the instrument carriage 130 of a manipulator arm. In the illustrated example, an ISA 300 is interposed between the instrument interface 242 and the carriage interface 310, although it should be appreciated that an instrument 120 may be coupled directly to an instrument carriage 130 in applications in which an ISA 300 is not needed.

Carriage drivers 320 on the carriage interface 310 are rotated by motors in the instrument carriage 130 to rotate adapter drivers 330 on the ISA 300 which, in turn, rotate instrument drivers 340 on the instrument 120. In the illustrated example, five sets of drivers are included, each controlling some aspect of manipulation and/or monitoring of the instrument 120. It should be appreciated that other examples may include more or fewer sets of drivers depending on the number of degree-of-freedom of the instrument 120 to be controlled. Furthermore, the placement of the drivers in relation to one another is not limited to the configuration illustrated. Two or more of the instrument drivers 340 may be rotationally coupled so that two or more motors may be used to drive a single function of the instrument 120. The carriage drivers 320 may each be operated in response to control signals from the control system 110 (FIG. 1). The control system 110 may operate each carriage driver 320 independently of the other carriage drivers. In some examples, a pair of carriage drivers 320 may be operated in a coordinated manner to control a degree-of-freedom on an instrument 120 in opposing directions. When two or more instrument drivers 340 are rotationally coupled, rotation of one carriage driver 320 and corresponding instrument driver 340 may cause rotation of one or more other instrument drivers 340 and corresponding carriage drivers 320.

Engagement features 325 on the carriage drivers 320 mate with corresponding engagement features 335l on the adapter drivers 330. Similarly, engagement features 335u on the adapter drivers 330 mate with corresponding engagement features 345 on the instrument drivers 340. In the illustrated example, the engagement features 325 on the carriage drivers 320 and the engagement features 345 on the instruments drivers 340 are in the form of pockets and the engagement features 335 on the adapter drivers 330 are in the form of corresponding bosses. However, any suitable arrangement of engagement features, not limited to pockets and bosses, may be used. In some examples, the engagement features 325 on the carriage drivers 320 and the engagement features 345 on the instruments drivers 340 may be in the form of bosses and the engagement features 335 on the adapter drivers 330 may be in the form of corresponding pockets. Furthermore, it should be noted that the number of engagement features on each driver is not limited to two as shown in the illustrated example but rather any suitable number of engagement features may be used.

Additional details of a backend assembly and, in particular, engagement of an instrument 120, ISA 300, and carriage 130 may be found in Int. Pat. App. Pub. No. WO 2015/142788 titled “METHOD FOR ENGAGING SURGICAL INSTRUMENT WITH TELEOPERATED ACTUATOR,” which is hereby incorporated by reference in its entirety.

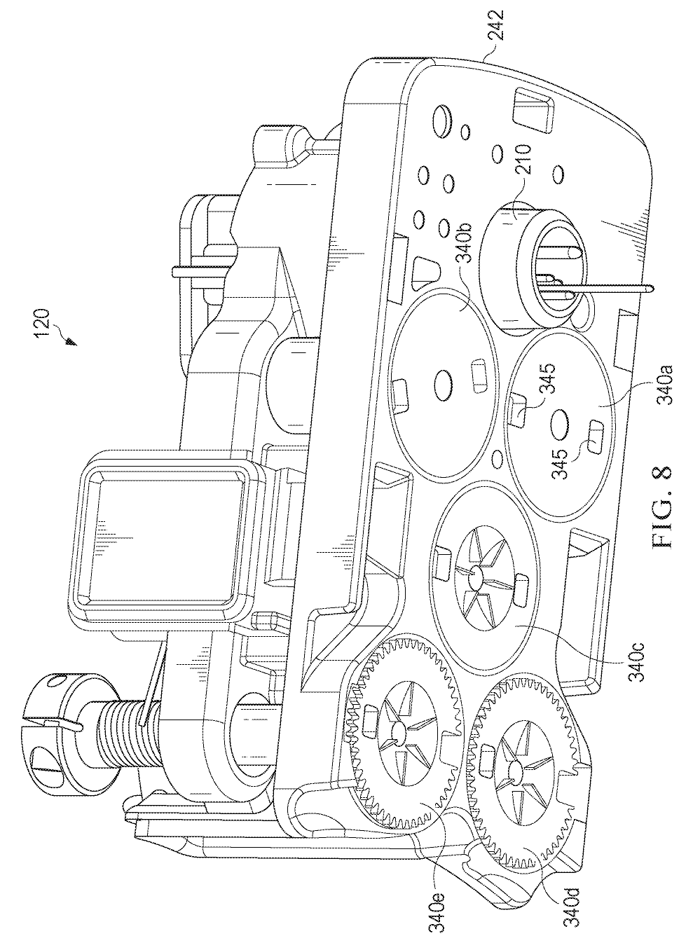

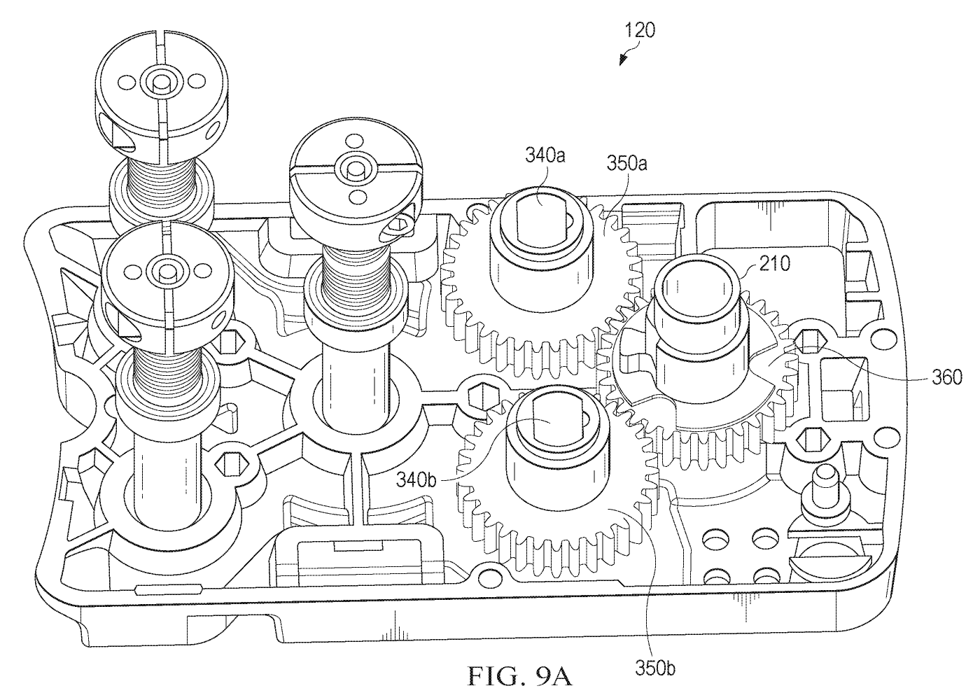

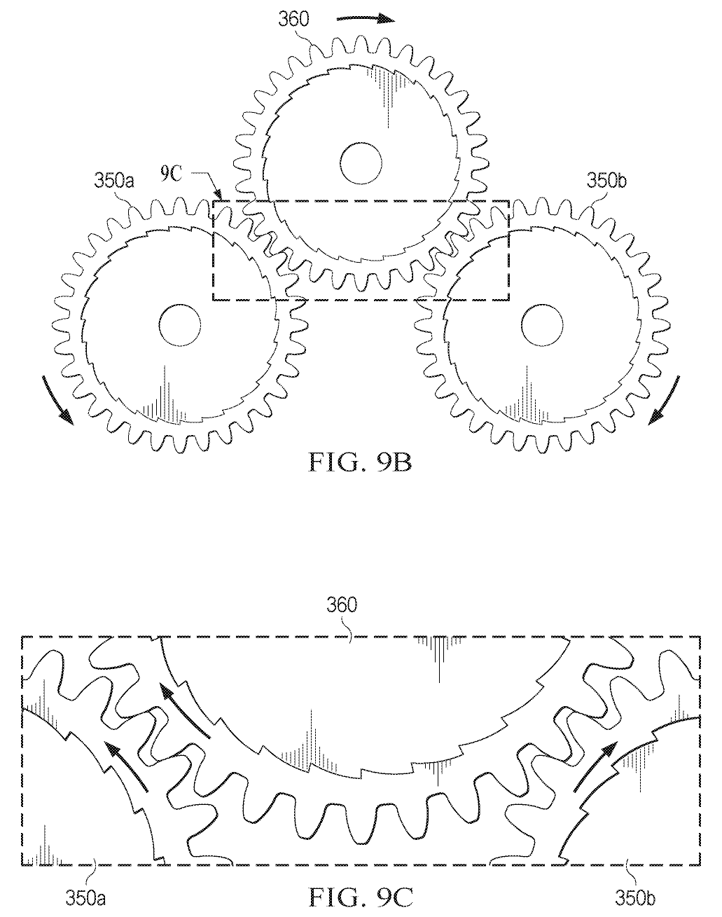

With reference to FIG. 8, the instrument interface 242 includes instrument drivers 340a-340e. In the illustrated example, instrument drivers 340a and 340b are configured as a pair for control of rotation of the shaft 210 (shown in truncated form for purposes of illustration) about its roll axis. The instrument drivers 340c-340e are configured for control of other movements of the instrument 120. For example, the instrument drivers 340c-340e may each be coupled to capstan, as shown in FIG. 9A, configured for winding of a cable that may extend through the shaft 210 to a wrist or an end effector. The description herein largely focuses on instrument drivers 340a and 340b, but it should be appreciated that the concepts described may be similarly applicable to instrument drivers 340c-340e.

As shown in FIG. 9A, which illustrates an internal portion of the proximal control mechanism 240 with a cover removed to avoid obscuring the illustrated internal components, each of instrument driver 340a and 340b are coupled to respective instrument drive gears 350a and 350b. Rotation of instrument driver 340a rotates instrument drive gear 350a, rotation of instrument driver 340b rotates instrument drive gear 350b, and vice versa. In some examples, the instrument drive gears 350a and 350b may be integral with the respective instrument driver 340. Instrument drive gears 350a and 350b engage shaft gear 360 which is coupled to the shaft 210. In this regard, when either of the instrument drivers 340a and 340b are driven by a corresponding carriage driver 320 (with or without an interposed adapter driver 330), the shaft gear 360 and shaft 210 are rotated, as is the other of the instrument drivers 340a and 340b. That is, rotation of the instrument driver 340a causes rotation of the shaft gear 360 which, in turn, causes rotation of the instrument driver 340b. Furthermore, the instrument 120 may be back-driven in that the shaft 210 may be rotated with respect to the proximal control mechanism 240 (e.g., manually by a user or due to friction between the shaft 210 and patient anatomy) which will, in turn, cause rotation of the instrument drivers 340a and 340b.

Mechanical design constraints, manufacturing tolerances, material properties, and various other considerations lead to backlash in the instrument drive system. For example, backlash may occur between one or more of a carriage driver 320 and an adapter driver 330, an adapter driver 330 and an instrument driver 340, an instrument driver 340 and an instrument drive gear 350, an instrument drive gear 350 and an instrument output (e.g., shaft gear 360), etc. Furthermore, cables and/or rods used to link an instrument driver 340 to a wrist 252 or an end effector 254 may flex and/or stretch leading to backlash.

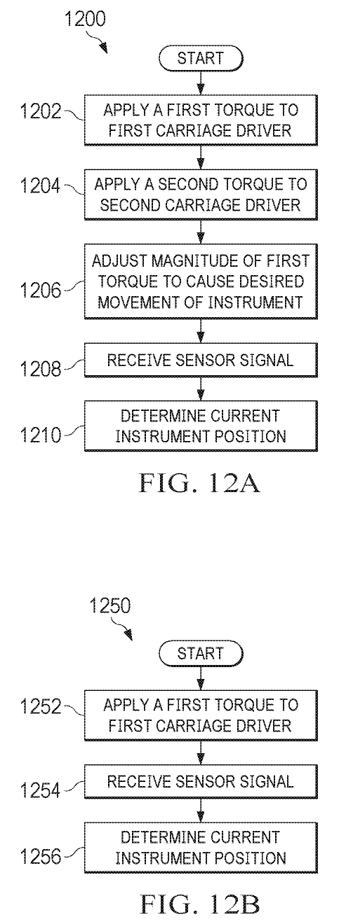

FIGS. 12A-12B illustrate examples of methods for determining an instrument position. In accordance with these methods, a sensor may be used to monitor an absolute position and/or rotational displacement of one or more components in the instrument drive system for the purpose of determining a position and/or orientation of the instrument 120. For example, an encoder may monitor a shaft of a motor configured to rotate the carriage driver 320b while another motor is driven to rotate carriage driver 320a. In another example, an encoder may directly monitor the carriage driver 320b. In some examples, the sensor monitors absolute position of a shaft. For example, the sensor may indicate the shaft is at a 30° position. Software in the control system 110 may maintain a record of shaft positions reported by the sensor to determine direction of rotation and when the shaft has crossed a 360° mark.

In method 1250 of FIG. 12B, at 1252 a first torque is applied to carriage driver 320a to cause rotation of the shaft 210 about its roll axis. Because the two carriage drivers 320a and 320b are rotationally coupled via the corresponding adapter drivers 330a and 330b, instrument drivers 340a and 340b, instrument drive gears 350a and 350b, and the shaft gear 360, movement of the shaft 210 caused by driving of the carriage driver 320a causes corresponding rotation of the carriage driver 320b which may be monitored with the sensor. At 1254, a sensor signal from the sensor associated with carriage driver 320b is received by the control system 110. At 1256, the control system determines a current position and/or orientation of the shaft 210 of the instrument 120 based on the sensor signal. For example, the sensor signal may indicate how many degrees the motor shaft has been rotated and in which direction. The control system 110 converts the magnitude and direction of the motor shaft rotation into a corresponding magnitude and direction of rotation of the shaft 210 to determine the current orientation of the shaft 210 and corresponding position of one or more points on the shaft.

However, backlash in the instrument drive system may lead to inaccuracies in the determination of the instrument position. That is, some degree of rotation of the shaft 210 may occur without corresponding rotation of the carriage driver 320a and/or carriage driver 320b. In order to reduce or compensate for backlash in the instrument drive system in the determination of instrument position and/or orientation (e.g., to accurately determine an instrument position and/or orientation, such as a roll position of the instrument shaft 210, despite presence of backlash), the control system 110 may be configured to apply a constant torque to one or more of the carriage drivers 320a, 320b to ensure that components in the instrument drive system remain in engagement in accordance with the method 1200 of FIG. 12A. For example, at 1202 the control system applies a first torque to a first carriage driver 320a to impart roll movement to the shaft 210. Depending on which direction of shaft roll movement is desired, the carriage driver 320a may be driven clockwise or counterclockwise. At 1204, in order to eliminate, reduce, and/or compensate for the effect of backlash on calculating a current position and/or orientation of the shaft 210, the control system 110 may simultaneously apply a constant second torque to the carriage driver 320b. The constant torque applied to carriage driver 320b will effectively bias the various components in the instrument drive system into continuous engagement with one another. This constant torque may be minimal in the sense that it may be effective to drive components into engagement with one another but may be of a lesser magnitude than the first torque, which is used by the control system 110 to roll the shaft 210. In some examples, the second constant torque may be applied to the carriage driver 320b only when a commanded motion of the shaft 210 is intended by the control system 110. That is, when the control system 110 prepares to direct rotation of the carriage driver 320a to rotate the shaft 210, the control system 110 may begin applying the second constant torque to the carriage driver 320b. In some examples, the second constant torque may be applied to the carriage driver 320b essentially always when the instrument is in use. That is, upon confirmation of installation of the instrument 120 on the carriage 130, the control system 110 may apply the second constant torque to the carriage driver 320b regardless of whether the control system 110 is actively controlling rotation of the shaft 210 with the carriage driver 320a. In yet additional embodiments, the application of the second constant torque can depend on a system mode of operation. For instance, if the instrument is an imaging device (e.g., an endoscope), the second constant torque can be applied when the system is in a camera control mode in which the control device(s) is configured to reposition or reorient the imaging device); if the instrument is a medical instrument, the second constant torque can be applied when the system is in a following mode in which the control device(s) is configured to reposition or reorient the medical instrument.

In some examples, inherent friction in the instrument drive system may exceed the magnitude of the constant second torque. In other words, even when no torque is being actively applied to the carriage driver 320a by its respective motor, the second constant torque may not be substantial enough to cause rotation of the shaft 210. In other examples, the constant second torque may be substantial enough to cause rotation of the shaft 210 and, in turn, rotation of the carriage driver 320a. In this regard, at 1206 the control system 110 may actively adjust the magnitude and direction of torque applied to carriage driver 320a to compensate for the constant second torque applied to the carriage driver 320b (as well as any external disturbance force acting on the shaft) to cause the desired movement of the shaft 210. For example, when it is desired that the shaft 210 remain still, the control system 110 may apply a first torque to the carriage driver 320a that is equal in magnitude but opposite in direction to constant second torque applied to the carriage driver 320b, assuming no external forces acting on the shaft. As a result, the net torque at shaft 210 is zero. Similarly, when it is desired to rotate the shaft 210, the control system 110 may apply a first torque to the carriage driver 320a that compensates for the constant second torque by increasing the first torque to counteract the second torque or decreasing the first torque to operate in conjunction with the second torque, depending on the desired direction of roll.

Due to the constant second torque being applied to the carriage driver 320b, the engagement features 325 of the carriage driver 320b are retained in contact with the engagement features 335l of the adapter driver 330b. In turn, the engagement features 335u of the adapter driver 330b are retained in contact with the engagement features 345 of the instrument driver 340b. In turn, the teeth of the instrument drive gear 350b are retained in contact with the teeth of the shaft gear 360.

Further, in some examples, the teeth of the shaft gear 360 are retained in contact with the teeth of the instrument drive gear 350a. In turn, the engagement features 345 of the instrument driver 340a are retained in contact with the engagement features 335u of the adapter driver 330a. In turn, the engagement features 335l of the adapter driver 330a are retained in contact with the engagement features 325 of the carriage driver 320a. However, given that the carriage driver 320a will frequently start, stop, and change direction of rotation to actively control the pose of the shaft 210, backlash may still occur on this side of the instrument drive system (e.g., between the carriage driver 320a and the shaft gear 360). Nevertheless, the effects of backlash are reduced on the other side of the instrument drive system (e.g., between carriage driver 320b and the shaft gear 360) due to the constant second torque.

As such the relative position and orientation between adjacent components in the instrument drive system is always, or nearly always, known such that error introduced into determining the orientation of the shaft 210 is reduced despite the presence of backlash in the instrument drive system. In particular, the relationship between a current orientation of the motor shaft driving carriage driver 320b, as measured by the sensor, is known with respect to the current orientation of the instrument shaft 210. In this regard, at 1208 a sensor signal is received from the sensor associated with the carriage driver 320b and, at 1210, a current position and/or orientation of the shaft 210 of the instrument 120 is determined based on the sensor signal.

FIGS. 9B-9C illustrate an example of how application of the constant second torque provides a known orientational relationship between adjacent components in the instrument drive system at any given time. Control system 110 may actively roll the shaft 210 of the instrument 120 by applying torque in a counterclockwise direction to carriage driver 320a. This results in counterclockwise rotation of the instrument drive gear 350a which, in turn, causes rotation of the shaft gear 360 in a clockwise direction. At the same time, the control system 110 applies constant second torque to carriage driver 320b in a clockwise direction. The arrows adjacent to the instrument drive gears 350a and 350b indicate the direction of torque applied by the respective motors whereas the arrow adjacent the shaft gear 360 indicates the resulting rotation of the shaft gear 360. The second constant torque effectively provides resistance to rotation of the instrument drive gear 350b. As a result, as shown in FIG. 9C, the gear teeth of the instrument drive gear 350b are retained in contact with the gear teeth of the shaft gear 360 and the gear teeth of the shaft gear 360 are retained in contact with the gear teeth of the instrument drive gear 350a. Because the constant second torque may be continuously applied, the control system 110 can reliably assume the gear teeth are in contact and that the effects of backlash between the gear teeth are minimized. This concept similarly applies to the respective engagement features between the carriage drivers 320, adapter drivers 330, and instrument drivers 340.

Assuming for example, that there is typically 2-3° of backlash in the instrument drive system (that is, the shaft 210 may roll 2-3° in either direction with no corresponding movement at the carriage drivers 320), the principles discussed herein may reduce the effects of backlash such that the shaft 210 may be rotated only 0-1° without causing corresponding movement at the carriage driver 320b (which is measured by the sensor). As a result, measurement of movement or position of the motor shaft at the carriage driver 320b by the sensor may be accurately and reliably translated into a corresponding orientation of the shaft 210 about its roll axis. In turn, the relative orientation between the instrument 120 and other instruments and/or the patient anatomy may be determined with improved accuracy and/or reliability.

When an endoscopic view of patient anatomy at a surgical site is being displayed to an operator on the operator input system 116 or display system 119 from an imaging element at the distal tip of the instrument 120, other instruments at the surgical site may be out of view. It is desirable to provide instrument position indicators on the display to indicate a position of out of view instruments. Examples of instrument position indicators are provided in U.S. Pat. App. Pub. No. 2017/0305016 titled “TOOL POSITION AND IDENTIFICATION INDICATOR DISPLAYED IN A BOUNDARY AREA OF A COMPUTER DISPLAY SCREEN” which is hereby incorporated by reference in its entirety. The techniques described herein, which improve reliability in calculating a current orientation of the shaft 210 (and, in turn, the current orientation of the endoscopic imaging element providing the real-time view of the surgical site) advantageously provide improved accuracy in placement of instrument position indicators for out of view instruments. In particular, the control system 110 may calculate a pose of the shaft 210 of the instrument 120b including the endoscopic image element and may further calculate a pose of one or more second instruments 120a, 120c, or 120d of the manipulator assembly 112 relative to the instrument 120b. This calculation may be based at least in part on a kinematic chain of the manipulator assembly 112 extending between the shaft 210 and the one or more second instruments.

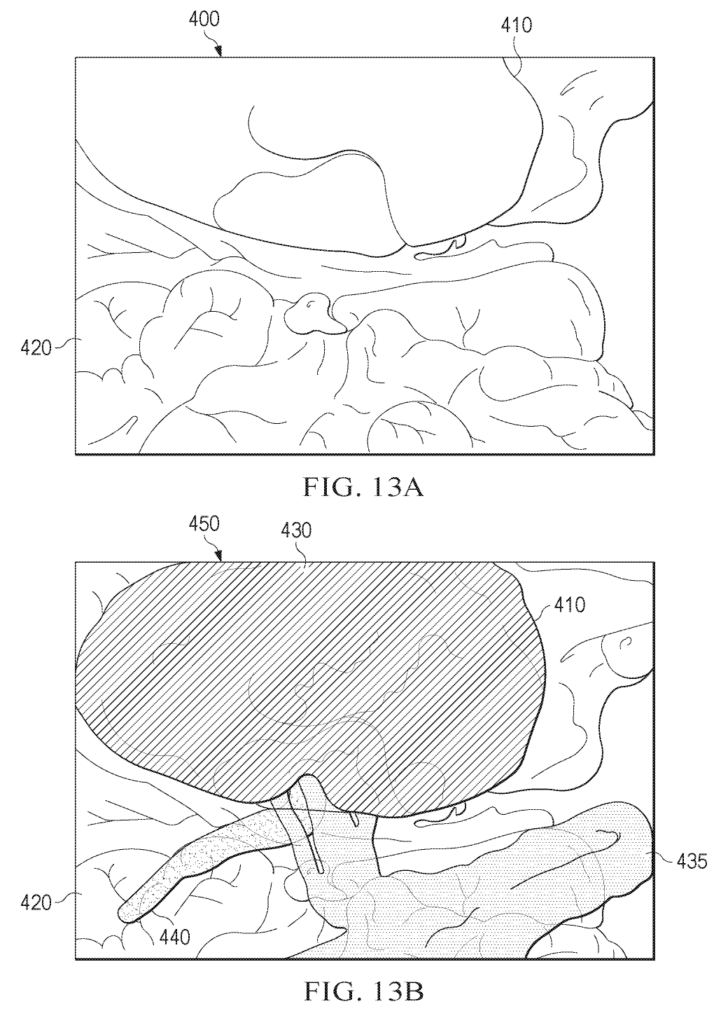

Additionally, it may be desirable to provide an augmented reality display in which graphical information is overlaid on a real-time endoscopic view. For example, a three-dimensional model of patient anatomy of interest may be generated from pre-operative imaging of the patient and displayed in juxtaposition with the real-time endoscopic view. FIG. 13A illustrates a real-time camera image 400 which may be provided by an imaging element of an instrument 120. The image 400 may include visible anatomical features which, in the illustrated example, include a kidney 410 and intestines 420. However, some anatomical features may be hidden from view of the operator, for example, embedded within or obscured by the visible anatomical features. Accordingly, an augmented reality image may be displayed in which at least a portion of a three-dimensional model is displayed with the image 400. As shown in FIG. 13B, augmented reality view 450 includes the visible anatomical features of the image 400 with additional augmented reality overlays which, in the illustrated example, include parenchyma 430, vasculature 435, and ureter 440.

It will be appreciated that assembling an augmented reality view in which model information is overlaid on a real-time image requires registering the real-time image to the model information. In some examples, a pose of the instrument 120 (and corresponding real-time image from the instrument) in an instrument reference frame may be registered to a surgical reference frame of the patient. A model reference frame of the model information may also be registered to the surgical reference frame such that the instrument reference frame is effectively registered to the model reference frame.

Inaccuracy in determining the current orientation of the endoscopic camera providing the real-time image may result in inaccurate registration and, in turn, graphical overlays may be improperly aligned with the real-time image. Moreover, a graphical overlay may tend to shift in space with regard to the underlying anatomy as the instrument is moved around. Accordingly, improved reliability in determining the current orientation of the shaft 210 of the instrument 120 as provided by the techniques discussed herein may provide for improved accuracy in registration of model information to the real-time image. Additional discussion of augmented reality views is provided in U.S. Pat. App. Pub. No. 2020/0093544 titled “SYSTEM AND METHOD FOR REGISTRATION AND COORDINATED MANIPULATION OF AUGMENTED REALITY IMAGE COMPONENTS” which is hereby incorporated by reference in its entirety.

It should be appreciated that although the concepts herein have been described in reference to movement of instrument shaft 210 about a roll axis, these concepts are similarly applicable to improving monitoring of other degrees-of-freedom of an instrument. For example, the principles discussed herein may be applied to carriage drivers and instruments drivers configured to control bending of a wrist 252 and/or manipulation of an end effector 254 in an X-direction and/or a Y-direction. Furthermore, although described in the context of rotational motors and drivers, the principles described herein are similarly applicable to linear actuators and drivers, for example, rack-and-pinion actuators.

In this description, elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom – e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention and that the embodiments of the invention are not limited to the specific constructions and arrangements shown and described, since various other modifications may occur to those ordinarily skilled in the art.

What is claimed is:

1. A robotically-assisted manipulator system, comprising:
an instrument including a first instrument driver and a second instrument driver, wherein the first and second instrument drivers are coupled such that movement of the first instrument driver causes movement of the second instrument driver;
an instrument manipulator including a first carriage driver, a second carriage driver, and a sensor configured to monitor a position of the second carriage driver, wherein the first carriage driver is configured to couple to the first instrument driver and the second carriage driver is configured to couple to the second instrument driver; and
a controller in operative communication with the instrument manipulator, the controller being configured to:
apply a first torque to the first carriage driver, the first carriage driver configured to impart motion to the first instrument driver to cause movement of the instrument in a degree-of-freedom;
apply a second torque to the second carriage driver;
adjust a magnitude of the first torque to cause a desired degree of movement of the instrument in the degree-of-freedom, wherein the magnitude of the first torque compensates for at least one of an external force on the instrument or the second torque;
receive a sensor signal from the sensor indicating a position of the second carriage driver; and
determine, based on the position of the second carriage driver, a current instrument position in the degree-of-freedom.

2. The robotically-assisted manipulator system of claim 1, wherein the controller is configured to apply the second torque to the second carriage driver when the desired degree of movement of the instrument in the degree-of-freedom is no movement.

3. The robotically-assisted manipulator system of claim 1, wherein the controller is configured to simultaneously apply the first torque to the first carriage driver and the second torque to the second carriage driver.

4. The robotically-assisted manipulator system of claim 1, wherein when the desired degree of movement of the instrument in the degree-of-freedom is no movement, the magnitude of the first torque is equal to a magnitude of the second torque.

5. The robotically-assisted manipulator system of claim 1, wherein the first and second carriage drivers each include a first engagement feature and the first and second instrument drivers each include a second engagement feature configured to mate with the first engagement feature of a corresponding one of the first carriage driver or the second carriage driver.

6. The robotically-assisted manipulator system of claim 5, wherein application of the second torque to the second carriage driver retains the first engagement feature of the second carriage driver in contact with the second engagement feature of the second instrument driver.

7. The robotically-assisted manipulator system of claim 1, wherein the instrument comprises a sterile adapter that comprises the first and second instrument drivers.

8. The robotically-assisted manipulator system of claim 1, wherein the controller is configured to continuously apply the second torque to the second carriage driver at a constant magnitude and to vary at least one of the magnitude or a direction of the first torque to impart the desired degree of movement of the instrument in the degree-of-freedom.

9. The robotically-assisted manipulator system of claim 1, wherein when the first torque and the second torque are applied in opposite directions, the magnitude of the first torque is greater than a magnitude of the second torque to impart the motion to the first instrument driver.

10. The robotically-assisted manipulator system of claim 1, wherein the controller is configured to reduce the magnitude of the first torque to equal a magnitude of the second torque to retain the instrument stationary in the degree-of-freedom.

11. The robotically-assisted manipulator system of claim 1, wherein the first instrument driver and the second instrument driver are coupled to each other by an output shaft of the instrument, each of the first and second instrument drivers being independently coupled to the output shaft.

12. The robotically-assisted manipulator system of claim 11, wherein the first instrument driver and the second instrument driver are rotationally coupled by the output shaft.

13. The robotically-assisted manipulator system of claim 12, wherein the first instrument driver, the second instrument driver, and the output shaft each comprise a gear.

14. The robotically-assisted manipulator system of claim 11, wherein the first instrument driver and the second instrument driver are translationally coupled by the output shaft.

15. The robotically-assisted manipulator system of claim 1, wherein the instrument is a medical instrument that includes a shaft having a distal end configured to be inserted into a patient anatomy.

16. The robotically-assisted manipulator system of claim 15, wherein an image capturing device is disposed at the distal end of the shaft.

17. The robotically-assisted manipulator system of claim 16, wherein the degree-of-freedom of the instrument is roll about a longitudinal axis of the shaft.

18. The robotically-assisted manipulator system of claim 17, wherein the controller is further configured to register a pose of the medical instrument in an instrument reference frame to a surgical reference frame.

19. The robotically-assisted manipulator system of claim 18, wherein the controller is further configured to calculate a pose of one or more second instruments of the manipulator system relative to the medical instrument.

20. The robotically-assisted manipulator system of claim 16, wherein the controller is further configured to register an image generated by the medical instrument to a model reference frame.

21. The robotically-assisted manipulator system of claim 20, wherein the controller is further configured to overlay model information on the image generated by the medical instrument.

22. The robotically-assisted manipulator system of claim 1, wherein the sensor comprises an encoder configured to monitor a position of the second carriage driver.

23. A robotically-assisted manipulator system, comprising:
an instrument including a first instrument driver and a second instrument driver, wherein the first and second instrument drivers are coupled such that movement of the first instrument driver causes movement of the second instrument driver;
an instrument manipulator including a first carriage driver, a second carriage driver, and a sensor configured to monitor a position of the second carriage driver, wherein the first carriage driver is configured to couple to the first instrument driver and the second carriage driver is configured to couple to the second instrument driver; and
a controller in operative communication with the instrument manipulator, the controller being configured to:
apply a first torque to the first carriage driver, the first carriage driver configured to impart motion to the first instrument driver to cause movement of the instrument in a degree-of-freedom;
receive a sensor signal from the sensor indicating a position of the second carriage driver; and
determine, based on the position of the second carriage driver, a current instrument position in the degree-of-freedom.
